(19) United States
(12) Patent Application Publication
(10) Pub. No.: US 2005/0221335 A1
(43) Pub. Date: Oct. 6, 2005

(54) Reporter gene

(76) Inventor: Anthony Kavanagh, County Dublin (IE)

Correspondence Address: JACOBSON HOLMAN PLLC, 400 SEVENTH STREET N.W., SUITE 600, WASHINGTON, DC, 20004, US

(21) Appl. No.: 10/865,909

(22) Filed: Jun. 14, 2004

Related U.S. Application Data
(63) Continuation of application No. PCT/IE01/00155, filed on Dec. 14, 2001.

(51) Int. Cl. C12Q 1/68

(52) U.S. Cl. 435/6

(57) ABSTRACT

Use of a gene which codes for a sialidase activity as a reporter gene is described. The gene may be used as a reporter gene in eukaryotic organisms or cells. A modified gene encoding a protein having sialidase enzymatic activity is also described. The nucleotide sequence encodes a prokaryotic or eukaryotic sialidase enzymatic activity preferably the nucleotide sequence encodes a Clostridium perfringens sialidase activity.

The invention relates to a novel reporter gene in particular a reporter gene for use in plants.

Reporter gene technology constitutes a powerful molecular tool for the analysis of gene expression and gene product localization in cells, tissues and whole organisms with applications in plant, animal, fungal and bacterial biotechnology.

A reporter gene acts as a readily detectable surrogate for the gene under study and is typically used as (a) a transcriptional gene fusion partner or (b) a translational gene fusion partner. When used as a transcriptional gene fusion partner, a reporter gene construct typically contains one or more regulatory elements from the promoter of the gene being analysed, fused with the structural sequence of the reporter gene, and sequences required for the formation of functional mRNA. When used as a translational gene fusion partner, a reporter gene construct typically contains the reporter gene fused in-frame with all or part of a second open reading frame (orf) which when translated results in the production of a fusion protein in which additional amino acid sequences are located at the N-terminal or C-terminal end (or both ends) of the reporter protein. Upon introduction of either type of reporter gene construct into cells or whole organisms, the expression levels of the reporter gene can be monitored either by detection of the reporter protein itself or by assay of the reporter protein's enzymatic activity. In addition, if the appropriate histochemical substrates are available, it may be possible to visualize the cell-specific or tissue-specific distribution of reporter protein activity. Sensitive reporter assays are necessary for the analysis of low levels of gene expression, for example when analysing transcriptionally weak promoters or when analysing gene expression in cells which transfect poorly.

In addition, a sensitive, easily assayable reporter gene can also be used (i) to facilitate the identification of (and in some cases the selection of) transformed cells and (ii) to produce marked transgenic organisms (e.g. plants) in which the reporter gene is physically linked to a gene coding for a trait that cannot itself be assayed enzymatically (e.g. a disease resistance gene). In such an application, detection of the reporter gene activity is used as an indirect assay for the linked gene.

The GUS reporter gene system, developed by Jefferson, Kavanagh and Bevan (1), revolutionised the analysis of plant gene expression and is currently the most widely used reporter gene system used in plants. The system is based on the uidA gene of E. coli which encodes the enzyme β-glucuronidase (GUS) and is used (i) in the analysis of plant gene promoter activity (i.e. as a transcriptional gene fusion partner) (ii) to investigate protein localisation in plant cells (i.e. as a translational gene fusion partner); (iii) as a easily assayable marker for the cosegregation of linked transgenes; (iv) in promoter trapping, enhancer trapping and gene trapping applications and (v) as a marker for the identification of genetically transformed cells or whole organisms.

The GUS reporter gene continues to be very widely used because it has the following characteristics:
(i) the GUS gene codes for an enzymatic activity which is essentially absent in plants i.e. there is little or no background endogenous GUS activity in plants;
(ii) there appear to be no natural endogenous substrates for GUS in plants;
(iii) for reasons (i) and (ii), when GUS is expressed in transgenic plants, it has no deleterious (i.e. toxic) effects on plant growth;
(iv) there are very user-friendly, highly sensitive, technically simple, inexpensive and rapid assays for GUS activity. Kinetic assays of GUS activity in tissue extracts are based on inexpensive, commercially-available calorimetric or fluorogenic substrates. The assays are also suitable for scale-up and automation;
(v) in particular, the availability of histochemical substrates allows the investigator to visualise the location of GUS activity (and hence gene activity) in plant tissues and cells. Hence the GUS reporter gene is ideal for the analysis (and identification) of genes that are expressed in a developmentally regulated manner or-are expressed in a tissue-specific or temporal manner;
(vi) the GUS enzyme is active over a wide range of pH, ionic and temperature values and is very stable both in vitro (in cell-free extracts) and in vivo; and
(vii) because of its stability and tolerance of assay conditions, the enzymatic activity of the GUS protein can also be readily detected in situ in polyacrylamide gels.

Although several other reporter genes have also been developed for use in plants, such as neomycin phosphotransferase (NPTII), luciferase (LUC), chloramphenicol acetyltransferase (CAT), beta-galactosidase (LacZ) and most recently the jellyfish green fluorescent protein (GFP), all have one or more disadvantages relative to GUS. For example, they require technically demanding or time-consuming or expensive assays or there may be problems with low sensitivity or it may be difficult or not possible to histochemically localise the reporter gene activity in cells and tissues.

For these reasons, there is currently no other reporter gene system that is equal to the GUS system, in terms of sensitivity, ease of use and the ability to localize the encoded enzymatic activity in cells, tissues and whole organisms. The usefulness and the applications of the GUS system would however be dramatically extended if a complementary reporter gene system could be developed with operational characteristics and properties at least on a par to those listed above (i)-(vii).

### STATEMENTS OF INVENTION

According to the invention there is provided the use of a gene which codes for a sialidase activity as a reporter gene. Preferably the gene codes for a sialidase activity for use as a reporter gene in an organelle or cell or organism having a eukaryotic-like transcriptional and translational system. Preferably the gene codes for a sialidase activity for use as a reporter gene in an organelle or cell or organism having a prokaryotic-like transcriptional and translational system. Most preferably the gene codes for a sialidase activity for use as a reporter gene in plants.

Preferably the gene of the invention which codes for a sialidase activity is used as an organelle-specific reporter gene.

Preferably the gene codes for a sialidase activity as a reporter gene in transiently transformed prokaryotic or eukaryotic cells. Most preferably the gene codes for a sialidase activity as a reporter gene in stably transformed prokaryotic or eukaryotic cells.

Preferably the invention provides the use of a gene of the invention wherein the nucleotide sequence of the natural gene is modified.

The invention also provides use of a gene of the invention wherein the gene comprises SEQ ID NO. 1.

Preferably the gene of the invention is a sequence-modified nanH. Most preferably the unmodified, natural gene is isolated from a prokaryotic or eukaryotic organism, a virus or a bacterium or from Clostridium perfringens.

One embodiment of the invention provides the use of a gene of the invention as a reporter gene in combination with another reporter gene. Preferably the other reporter gene is the uidA gene of Escherichia coli which encodes the enzyme β-glucuronidase (GUS).

The invention also provides a gene having SEQ ID NO. 1.

The invention further provides a modified gene encoding a protein having sialidase enzymatic activity.

Preferably the nucleotide sequence of the modified gene encodes a prokaryotic or eukaryotic sialidase enzymatic activity. Most preferably the nucleotide sequence of the modified gene encodes a bacterial sialidase enzymatic activity.

In one embodiment of the invention the nucleotide sequence of the modified gene encodes a Clostridium perfringens sialidase activity.

The invention further provides a modified gene having SEQ ID No.1 the nucleotide sequence of which is based on the nanH gene from Clostridium perfringens which codes for a sialidase enzymatic activity.

The invention also provides a sialidase enzymatic activity encoded by a gene of the invention. Sialidase enzyme activity is defined as an enzymatic activity that releases sialic acid residues or derivatives thereof from naturally occurring sialoglycoconjugates such as for example glycoproteins, glycolipids and from synthetic substrates such as for example 4-methylumbelliferyl-alpha-D-N-acetlyneuraminic acid.

Preferably the invention provides use of a gene of the invention or a derivative thereof in the production of sialidase. Most preferably the gene or a derivative thereof is used in the production of anti-sialidase antibodies or in the production of a vaccine.

Preferably the gene of the invention or a derivative thereof is used to investigate the biological consequences of sialoglycoconjugate cleavage in animals. Most preferably the gene of the invention or a derivative thereof or the encoded protein is used as a therapeutic agent in animals including man.

The gene of the invention or a derivative thereof may also be used in the production of transgenic plants or animals and transgenic plant or animal cell lines, for quantifying or detecting sialidase (NAN) activity in cell-free extracts or in whole organisms, organs, tissues or cells or in histological sections thereof or in vivo in whole cells using fluorogenic, chromogenic or calorimetric substrates or for simultaneously quantifying or detecting NAN and GUS activity in cell-free extracts or in whole organisms, organs, tissues or cells or in histological sections thereof or in vivo in whole cells using fluorogenic, chromogenic, colorimetric or histochemical substrates.

The following biological material has been deposited with NCIMB in compliance with the requirements of the Budapest Treaty on the International Recognition of Microorganisms for the purpose of Patent Procedure.

| Deposited Material | Deposit Date | Accession Number |
|---|---|---|
| Synthetic mnanH gene | Nov. 2, 2001 | NCIMB 41120 |

### BRIEF DESCRIPTION OF THE DRAWINGS

The invention will be more clearly understood from the following description thereof given by way of example only with reference to the accompanying figures in which:

FIG. 1 gives the nucleotide sequence ID NO. 1 of the resynthesised and sequence-modified nanH gene (abbreviated as mnanH). The numbered lines show the nucleotide sequence of the synthetic mnanH gene. The line immediately above each numbered line shows the predicted amino acid sequence encoded by mnanH. The line immediately below each numbered line shows the corresponding nucleotides found in the natural, unmodified nanH gene of Clostridium perfringens (2) (GenBank Accession: Y00963) that were altered in mnanH. The single modification of the protein sequence (amino acid residue 75, D→E) is highlighted in bold;

FIG. 2 shows a comparison of pH-activity profiles of the mNAN and native NAN proteins. Enzymatic activities were measured using the substrate MUN in a range of 50 mM sodium citrate buffers;

FIG. 3 shows the construction of vectors for transformation of plants with mnanH under control of either the rbcS or the CaMV 35S promoters. First, the assembled mnanH gene (A) was excised from pUC19-mnanH using Xba I and Sac I and placed downstream of the CaMV and rbcS promoters in pROK219 and pROK8, respectively (B). The promoter-mnanH fragments were then excised using Hind III and Sac I and ligated into the corresponding sites in pBIB-HYG, which co-transfers a hygromycin resistance gene (hpt) into tobacco. (3′ nos: nopaline synthase transcriptional terminator; B_{R} and B_{L} are the T-DNA borders.);

FIG. 4 shows the transient expression of mNAN and GUS in tobacco and onion cells. Two pUC19-based vectors containing either the mnanH or the GUS gene under control of the CaMV promoter were precipitated onto separate batches of 1.1μ tungsten microparticles which were then mixed together prior to bombardment. Tobacco leaf (A) and onion epidermis (B) stained with X-NeuNAc and X-GlucM after bombardment. Cells expressing GUS- are stained magenta (pink) (arrowed black) while cells expressing mNAN are stained blue [remaining cells], cells expressing both activities are indicated with an open-headed arrow;

FIG. 5 shows the detection of mNAN and GUS activities in situ in a polyacrylamide gel following electrophoretic separation of total protein samples prepared from transgenic tobacco plants. The location of GUS and mNAN activity bands in the gel are indicated. Total protein extracts were prepared in GEB buffer from transgenic plants containing the following constructs:
lane 1: CaMV-GUS; 2: CaMV-GUS and CaMV-mnanH; 3: CaMV-mnanH;

FIG. 6 shows the histochemical detection of mNAN activity in A: the root of a whole transgenic seedling transformed with CaMV-mnanH; B: individual root cells of the seedling shown in A; C: shows a stained and unstained pollen grain from a transgenic tobacco line transformed with rbcS-mnanH;

FIG. 7 shows the histochemical detection of mNAN and/or GUS activities in transgenic seedlings containing the following constructs: A: CaMV-mnanH; B: P20-GUS; C: P20-GUS×CaMV-mnanH; In A, high levels of mNAN activity are localised in the root; in B, high levels of GUS activity are localized in the cotyledons; in C, an F1 hybrid seedling resulting from a cross between the P20-GUS and CaMV-mnanH parental lines shows GUS activity in the cotyledons and NAN activity in the root;

FIG. 8 shows the histochemical detection of both mNAN and GUS activities in different cells in a transverse tissue section across a node of a Bmy-GUS×CaMV-mnanH transgenic plant, S: stem; P: petiole;

FIG. 9 is a graph showing the determination of mNAN and GUS activity using the fluorogenic substrates MUG and ReG, respectively. NAN1, GUS1 represent the reaction rates obtained when each enzymatic activity was measured independently i.e. with one substrate at a time in separate assay tubes; NAN2, GUS2 represent the reaction rates obtained when both activities were measured simultaneously i.e. with both substrates present in the same assay tube;

FIG. 10 shows the structure of two gene fusion constructs encoding mNAN-GFP or GFP-mNAN fusion proteins. In construct A: pROK219-NG the encoded fusion protein contains mNAN at the N-terminus and GFP at the C-terminus whereas in construct B:pROK219-GN mNAN is located at the C-terminus of the encoded fusion protein. In each construct, the mNAN and GFP open reading frames (orfs) are joined by a peptide linker, the amino acid sequence of which is shown. ATG signifies the translational initiation codon for the fusion proteins. These vectors were used directly for transient expression in plant cells; and

FIG. 11 Demonstration of mNAN and GFP activities in onion cells bombarded with the constructs pROK219-NG and pROK219-GN. A and B pROK219-NG, panel A shows the encoded fusion protein's mNAN activity detected histochemically and panel B shows its green fluoresence activity detected by UV epifluoresence microscopy; C and D: pROK219-GN, panel C shows the encoded fusion protein's mNAN activity detected histochemically and panel D shows its green fluoresence activity detected by UV epifluoresence microscopy.

### DETAILED DESCRIPTION

The present invention relates to a novel, alternative, highly sensitive reporter gene system that can be used alone or in combination with the GUS reporter gene or other reporter gene systems.

The invention will be more clearly understood from the following description thereof given by way of example only.

The invention relates to the total synthesis of a bacterial gene, the nucleotide sequence of which was modified to enable its use as a novel, highly sensitive reporter gene in plants and other organisms. The gene codes for a sialidase enzyme (syn: neuraminidase) similar to that encoded by the nanH gene of Clostridium perfringens (1). Sialidases have been identified in many animal lineages (Echinodermata through Mammalia) and in diverse microorganisms but they and their sialyl substrates, have not been found in plants. For this reason therefore, in principle, a gene encoding a sialidase activity ought to constitute (assuming it possesses the appropriate criteria as discussed above) an ideal reporter gene for use in plants. However, our attempts to express the native unmodified nanH gene in plants were unsuccessful. In contrast, the synthetic sequence-modified gene, called modified nanH (mnanH), was found to be very efficiently expressed in plant cells. Moreover, mnanH can be used as a novel reporter or marker gene not only in its own right but also in combination with the GUS reporter gene or any other reporter gene. The enzyme encoded by the mnanH gene possesses a more than 3-fold higher specific activity than the GUS enzyme. Consequently, its sensitivity as a reporter enzyme is potentially (and actually) far greater than that of the GUS enzyme. The mnanH reporter gene therefore offers the possibility of detecting very low levels of gene expression (e.g. levels directed by very weak promoters), levels that might not be detectable with the GUS system.

The mnanH gene of the present invention has been optimised for expression in plant cells. However it is expected that the gene would also be expressed at high level in other eurkaryotic cell types (eg. insect, nematode, fungal, mammalian) and in prokaryotic cells. Therefore it is expected that the gene of the present invention could be used not only to create model transgenic plants but also transgenic animal systems which could for example be used to investigate sialoglycoconjugate metabolism and the biochemical, physiological or phenotypic consequences of sialidase-mediated cleavage of sialic acid residues from the oligosaccharide components of glycoproteins and glycolipids. Furthermore, because of their hydrolytic activity towards sialoglycoconjugates, sialidases have several important therapeutic applications (3). Thus, silaidases are useful (a) in treating or preventing inflammation and inflammatory disorders (e.g. rheumatoid arthritis or Crohn's disease) and (b) treating or preventing pulmonary disorders characterized by an overproduction or excess of mucus (e.g. cystic fibrosis). The gene of the present invention could therefore have potential as a gene therapeutic agent in respect of these conditions.

The mnanH gene has the following advantages over the previously described reporter genes used in plants.
it encodes an enzyme activity not present in plants;
no natural substrates of the encoded enzyme are present in plants;
the encoded protein is non-toxic in plants;
the encoded enzymatic activity can be detected using low-tech, specific, rapid, inexpensive and highly sensitive assays based on available colorimetric and fluorogenic substrates; and
histochemical substrates are also available which allow the visualisation of the location of gene activity in plant tissues.

Throughout the specification the term “modified gene” refers to any genetically modified derivative of a naturally occurring (natural or native) gene in which the nucleotide sequence has been altered (relative to the naturally occurring gene) in order to facilitate or optimise its expression in target organisms other than (and including) the organism from which the natural gene was originally isolated or in order to confer novel enzymatic properties on the encoded protein. A modified derivative of a natural gene might be constructed by, for example only and not by way of limitation, (a) modifying the nucleotide sequence of the natural gene to facilitate its efficient transcription or to ensure that the mRNA transcript is efficiently translated (e.g. by codon-optimisation) (b) modifying the nucleotide sequence of the natural gene in such a way as to (i) alter the amino acid sequence of the encoded protein or (ii) to add additional amino acid sequences to the N-terminus or C-terminus of the encoded protein (i.e. N- or C-terminal extensions) (c) modifying the nucleotide sequences located upstream (5′) and downstream (3′) relative to the coding sequences in order to improve transcription rates or in order to improve mRNA stability and/or translational efficiency or (d) insertion of an intron or introns into the coding sequence for the reasons outlined in (c) or in order to prevent expression of the gene in prokaryotic organisms while permitting expression in eukaryotic organisms.

### Experimental Procedures

Methods of Construction of the Synthetic mnanH Gene

Three different approaches were taken to construct three sections of the modified gene, mnanH. The first section, from BamH I to Apa I, was constructed from six 5′-phosphorylated PAGE-purified 80-mers (Genemed Biotechnologies, Inc., Ca.). The oligos (75 pmol) were annealed in pairs in a solution of 50 mM Tris-Cl (pH 7.9), 100 mM NaCl and 10 mM MgCl₂, by heating to 90° C. and allowing to cool slowly to room temperature, creating 8 base 5′ overhangs. Two sets of annealed oligos (15 pmol) were then ligated in T4 ligase buffer with three units T4 ligase at 20° C. overnight, purified from a 10% acrylamide gel, and then ligated to the remaining pair. In fact, it was necessary to amplify the full-length annealed product by PCR with flanking primers (4). The second section, from Apa I to Afl II, was constructed in a similar fashion by annealing eight 110-mer oligonucleotides in complementary pairs, each of which possessed 20 base single-stranded ends capable of ligating with another pair (5). It was necessary in this case also to PCR amplify the ligated annealed products with flanking primers before cloning. The third section, from Afl II to Sac I, was assembled according to the method of Stemmer et al. (6), using 28 standard quality 40-mers (MWG, Germany) which when annealed in pairs possessed 20 base 3′ overlaps. A 590 bp fragment was constructed by PCR extension of overlapping oligonucleotides, which form larger DNA chains with each round of polymerisation, followed by amplification of the full-length product by a second round of PCR with flanking primers. The mnanH gene sequence was adjusted so that the G+C content of each of the 20 base single-stranded overhangs was at least 35%.

Enzyme Kinetic Measurements

NAN (and mNAN) assays were carried out in solution using 2′-(4-methylumbelliferyl)-α-D-N-acetylneuraminic acid (MUN, Sigma) as substrate. GUS activity was measured using the substrates 4-methylumbelliferyl β-D-glucuronide (MUG, Sigma) or resorufin β-D-glucuronide (ReG, Sigma). Triplicate reactions were typically carried out in a volume of 0.5 ml GEB at 37° C. for 60 min, and 50 μl aliquots were removed at intervals, the reaction terminated by adding the aliquot into 3 ml 0.33M Na₂CO₃, and the reaction products (methylumbelliferone (MU) or resorufin) measured against appropriate standards on a Perkin Elmer LS-50B Luminescence Spectrophotometer (excitation at 365 nm, emission at 445 nm for MU and 571/585 nm for resorufin). Kₘ and Vₘₐₓ values were determined according to the Michaelis-Menten equation by assay with substrate concentrations increasing from 0.1 mM to 1.0 mM, and a minimum substrate to enzyme molar ratio of 10⁶. Protein concentrations were determined by the Bio-Rad (Herts., UK) microassay.

Histochemical Assay

Tissue sections and whole seedlings were stained for mNAN and GUS activity using 5-Bromo-4-chloro-3-indolyl-β-D-N-acetyl neuraminic acid (X-NeuNAc, Rose Scientific, Ohio) and 5-Bromo-6-chloro-3-indolyl-β-D-glucuronide (X-GlucM, Glycosynth, Cheshire, UK), which, upon enzymatic cleavage, release blue and magenta chromophores, respectively. Reactions were typically carried out in GEB with 1 mM potassium ferrocyanide, 1 mM potassium ferricyanide, and 1 mM X-GlucM and/or 0.5 mM X-NeuNAc. Tissue samples were first washed briefly in GEB and then substrates were introduced by vacuum infiltration for 1 min and reactions carried out for 20 min to overnight at 37° C. Chlorophyll was then removed from samples by soaking first briefly in 50% ethanol and then overnight in 80% ethanol. Photographs were taken using a digital Olympus DP10 camera mounted on an Olympus SZX9 light microscope.

Expression of mNAN and GUS Enzymes in E. coli

The synthetic mnanH gene and GUS reporter gene were cloned into pET3a and pET24d vectors (Novagen, Wisconson), respectively, for high-level expression from the T7 promoter. mnanH was modified by PCR to place an Nde I site at the translation start site and a BamH I site immediately after the stop codon and cloned into pET3a cut with Nde I and BamH I. GUS was cloned directly into the Nco I and EcoR I sites of pET24d. The constructs were transformed into E. coli BL21 (DE3) and cultured initially in volumes of 2 ml Terrific Broth (TB) containing 200 μg/ml carbenicillin (NAN) or 50 μg/ml kanamycin (GUS). When these cultures had grown to OD 0.5, 0.3 ml aliquots were spun down and the cells resuspended in 10 ml fresh TB, containing 1 mM IPTG and 500 μg/ml carbenicillin or 50 μg/ml kanamycin as appropriate. Following overnight growth, soluble cell protein was extracted by first centrifuging the cells, resuspending them in 1/20^{th} volume of 20 mM TrisCl (pH 7.5) containing 100 μg/ml lysozyme and incubating for 15 min at 30° C. The mixture was then sonicated on ice (30 bursts for 5 seconds at 50% duty), centrifuged at 11,500 rpm for 10 min, and the supernatant analysed by SDS-PAGE. The amount of mNAN and GUS protein in the extracts was determined by comparison with equivalent size protein markers of known concentration. Kₘ and Vₘₐₓ values were determined for both mNAN and GUS as described above (Enzyme kinetic measurements).

Transient Expression in Plants

Transient expression studies were performed in tobacco leaf and onion epidermis using the Biolistic PDS-100/He Particle Delivery System (Bio-Rad, Herts., UK). DNA was prepared using a Quiagen Plasmid Midi Kit (Quiagen, Crawley, UK) and quantified by spectrophotemetry. 1.1μ tungsten microcarriers were washed and 3 mg coated with 5 μg plasmid DNA according to the manufacturer's protocol. A tissue sample was placed on a 1% agar plate at a distance of 9 cm from the microcarrier launch assembly. Macrocarriers were loaded with the DNA-coated microcarriers, set at a distance of ¼ inch from the rupture disk (rupture pressure 1100 psi), and bombardments were carried out under a vacuum of 28 inches Hg. The petri dish was then sealed and the tissue incubated at room temperature for 24-48 h before histochemical or microscopic analysis.

Plant Transformation

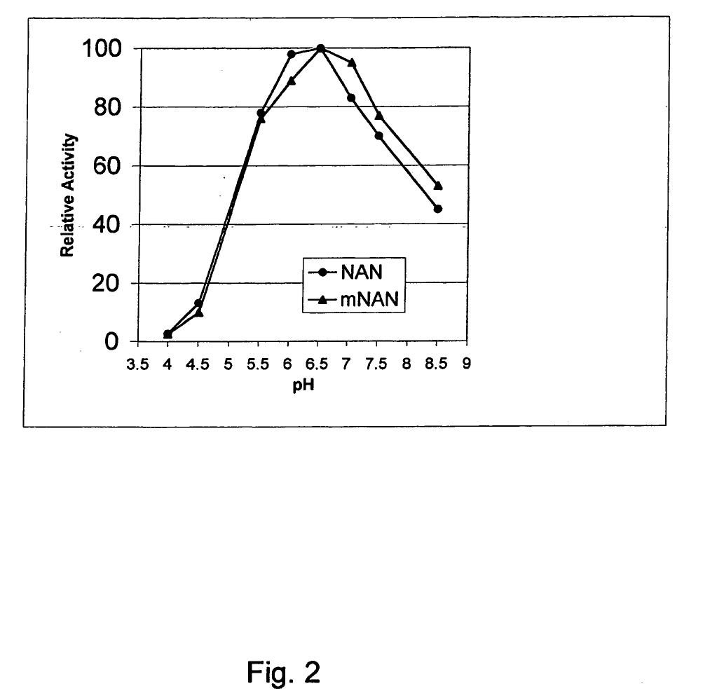

pBIB-HYG vectors carrying mnanH with appropriate promoter and terminator (FIG. 2) were introduced into competent Agrobacterium tumefaciens LBA4404 cells by heat shock as described (7). Plasmids were then confirmed in Agrobacterium by preparation according to the method of Holmes and Quigley (8) and analysis by restriction endonuclease. Leaf strips of N. tabacum, var. Samsun were transformed by incubation with A. tumefaciens and selection on NBM and MS agar (9) containing 40 μg/ml hygromycin.

Simultaneous Quantitation of NAN and GUS Activities

Since both mNAN and GUS can be assayed in the same buffer (GEB), it is convenient to prepare plant extracts in GEB and assay their activities in parallel using MUN and MUG. The possibility of detecting both enzyme activities in the same reaction was also investigated using resorufin β-D-glucuronide (ReG, Sigma) as a GUS substrate and MUG for detection of mNAN. Because enzymatic cleavage of both substrates releases a fluorogenic product with substantially different spectral properties, the two activities can in theory be quantified in the same sample using a fluorimeter equipped with appropriate filters. The Kₘ for GUS activity using ReG as substrate was determined to be 0.15 mM and this concentration was used in subsequent reactions. Extracts containing both activities were assayed for mNAN and GUS independently in separate reactions and also simultaneously in the same reaction.

In situ Detection of GUS and mNAN Enzymatic Activities in Polyacrylamide Gels Following Electrophoretics Separation

GUS and mNAN activities were detected in polyacrylamide gels. The first method involved separation of the enzymes by non-denaturing polyacrylamide gel electrophoresis (PAGE). The second involved separation of the enzymes by denaturing sodium dodecyl sulphate (SDS)-PAGE (with sample denaturation at 60° C.) followed by a subsequent renaturation treatment (10). Non-denaturing mini-gels comprising 8% polyacrylamide, 0.375 M TrisCl (pH 7.5), 0.1% ammonium persulphate, and 0.05% TEMED were prepared without a stacking gel. Running buffer and samples were prepared as described (11) and gels were electrophoresed at 140V for approximately 3 hours. Gels were soaked in GEB for 10 min at room temperature and then incubated at 37° C. for approximately 30 min in GEB containing 0.1 mM MUG and 0.05 mM MUN. Fluorescent MU bands were photographed on a UV transilluminator using Polaroid 665 film and a Wratten 2E filter.

### Results and Discussion

Synthesis of a nanH Gene Modified for Optimal Expression in Plants.

Attempts to express the native (i.e. the natural, unmodified) small nanH gene from Clostridium perfringens A99 in plant cells were not successful most probably because of the occurrence throughout the gene of AT-rich sequences capable of functioning as cryptic polyadenylation signals in eukaryotic cells. For this reason, it was decided to construct a sequence-modified synthetic gene, designated mnanH.

Criteria for sequence-modification of the nanH gene. The following criteria (reviewed in: Koziel et al., 1996 (12)) were employed in modifying the nucleotide sequence of the native C. perfringens nanH gene with a view to optimizing translational efficiency and mRNA stability and avoiding detrimental post-transcriptional processing events in eukaryotic cells e.g. polyadenylation:
mnanH codons were selected based on usage frequencies in Nicotiana tabacum and Arabidopsis thaliana.
the overall G+C content of the mnanH coding sequence was increased from 31.8% (in the native Clostridium perfringens nanH gene) to 43.8%, based on G+C content in highly expressed plant genes (13).
Polyadenylation signal sequences e.g. AATAAA, or similar hexamers with a one base mismatch which occur frequently throughout the native nanH orf, and TTTGTA were not included in the sequence of mnanH (14).
ATTTA strings, which are associated with mRNA instability (15) were not included in mnanH.
No sequences with the potential to form significant mRNA stem-loops (stem length≧10;≧22 bonds) were included in mnanH.
Potential intron splice sites were avoided in the modified sequence.
For improved initiation of translation, the sequence AACA was positioned 5′ to the ATG initiation codon in mnanH in accordance with the reported plant consensus (16). A second version of the mnanH gene was constructed with the initiation consensus sequence GCT positioned 3′ to the ATG codon, but no increase in NAN enzymatic activity was observed in tobacco (data not presented).

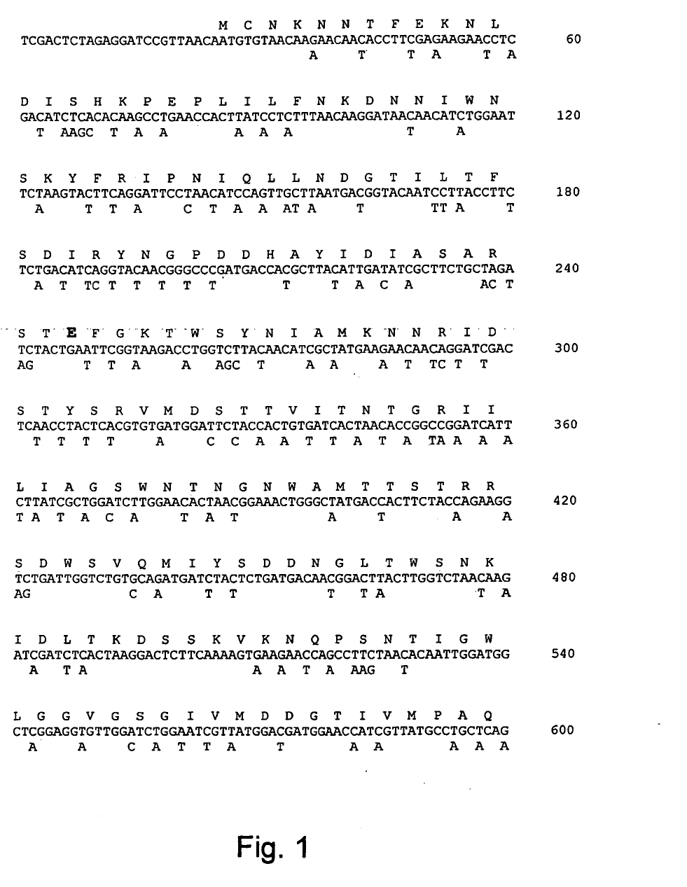

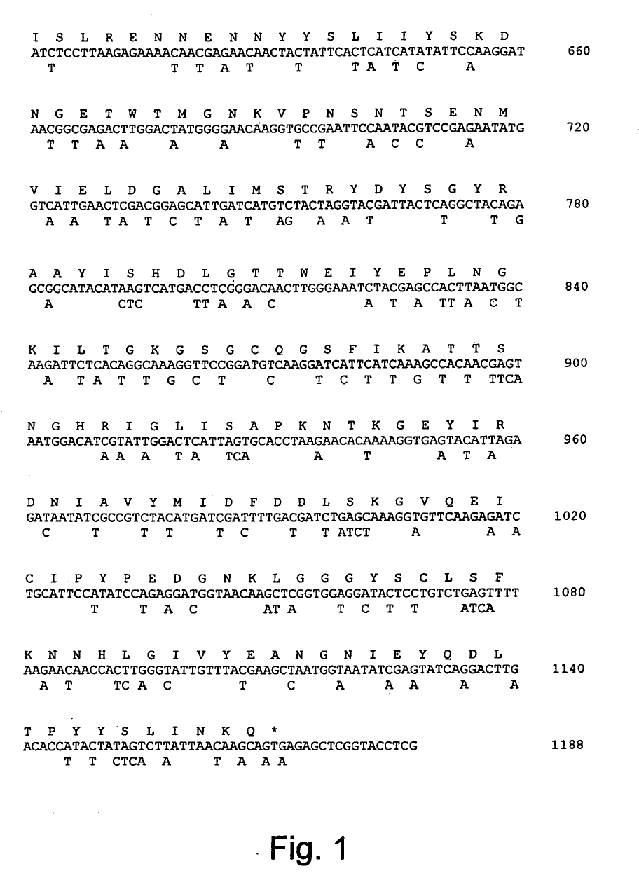

Of the three construction methods used, the PCR synthesis method of Stemmer et al. (6) was the most effective, based on the error-rate, expense and time-consumption involved. The sequence of mnanH is shown in FIG. 1.

Determination of Optimal Assay Conditions for Measuring mNAN Activity

The activity of mNAN was determined under different assay conditions using sonicated extracts of E. coli harbouring mnanH-pROK219 as the enzyme source (as described). Initially, activities were compared at pH 5.7 between different buffers (sodium acetate, sodium phosphate or sodium citrate) and ionic strengths (50, 100, 200 mM). A buffer containing 50 mM sodium phosphate was found to give optimal activity. The pH profile of mNAN activity was also measured in a range of 50 mM sodium citrate buffers, from pH 4.0 to 8.5, and compared to the native NAN enzyme (FIG. 2). mNAN was active over a wide range of pH values (as was native NAN), with optimal activity at around pH 6.5-7.0, which represents a slight shift towards alkaline conditions relative to native NAN activity. The combined effects of increasing the pH value of the 50 mM sodium phosphate buffer to pH7 and of incorporating the additional components used in GUS Extraction Buffer (GEB: 50 mM sodium phosphate (pH 7.0), 1 mM EDTA, 0.1% Triton X-100, and 10 mM β-mercaptoethanol, (modified from Jefferson et al., (1)) were also investigated. The results showed that mNAN can be extracted and assayed in GEB with no significant compromise of its performance (data not shown).

Determination of mNAN and GUS Relative Specific Activities.

In order to determine the relative specific activities of mNAN and GUS, each enzyme was expressed at high level in E. Coli. Using protein extracts containing known amounts of each enzyme, mNAN and GUS Vₘₐₓ values for the substrates MUN and MUG, respectively, in GEB were determined to be 3370 (mNAN) and 998 (GUS) μmol MU/min/mg protein. The corresponding mNAN and GUS Kₘ values (for their respective substrates MUN and MUG) were 0.20 and 0.17 mM respectively. Thus, mNAN and GUS display similar substrate affinities for these substrates, but the specific activity of mNAN is approximately 3.4 times greater than that of GUS.

Transient Expression of mnanH and GUS Genes in Tobacco and Onion

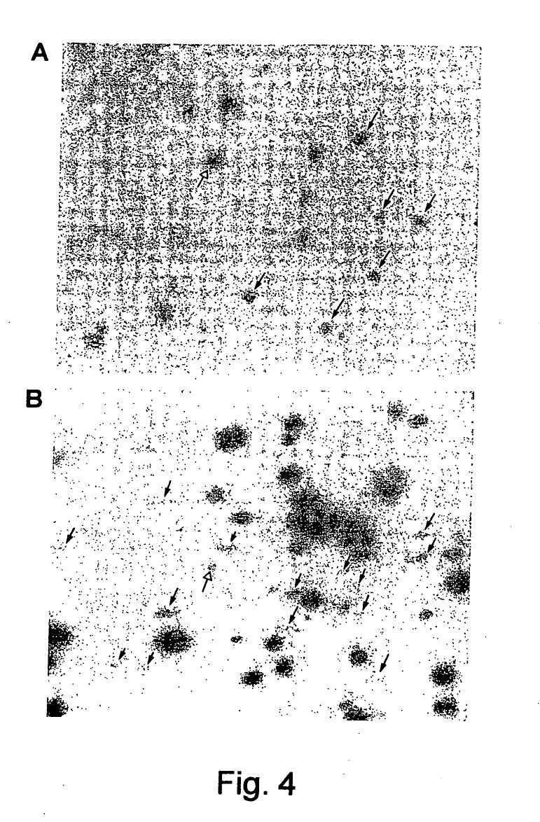

For transient expression of mNAN and GUS in tobacco leaf and onion epidermal cells, the vectors pROK219-mnanH (FIG. 2) and pGUS-HYG (a similar pUC19-based vector containing a CaMV35S-GUS gene casette) were used. DNA from each of the vectors was precipitated onto a separate batch of tungsten microparticles which were then mixed prior to bombardment. Following bombardment, tissue samples were incubated for 48 h and then stained with the histochemical reagents X-GlucM and X-NeuNAc overnight. Thus, cells expressing mNAN and GUS stained blue and magenta, respectively, and cells which express both of the enzymes stained purple (FIG. 4). In onion cells mNAN, but not GUS, was detected using 0.1 nM substrate, while both activities were clearly visible when 1 mM substrate was used. It is inferred from this that GUS has a higher Kₘ for X-GlucM than mNAN has for X-NeuNAc. In practice, transient expression and detection of GUS and NAN in tobacco and onion cells is conveniently achieved using the same bombardment and staining conditions.

Expression of mnanH Transgenes in Stably Transformed Tobacco Plants

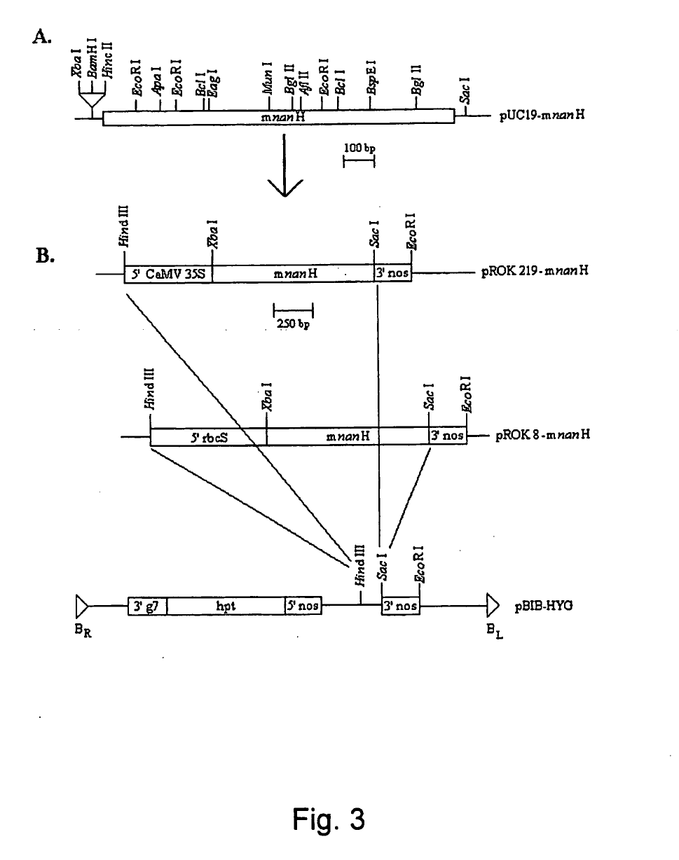

Stable transformations of N. tabacum var. Samsun were performed with mnanH in pBIB-HYG vectors under control of the CaMV 35S and rbcS promoters (FIG. 3). The specific activity of mNAN using MUN as the substrate was determined in cell-free extracts of leaf and root tissue from young primary transformant plants. A typical example of the activity values obtained is shown in Table 1 below. In transgenic plants in which mnanH expression was directed by the light-regulated rbcs promoter, mNAN activity in leaves as expected, was typically more than 6-fold higher than in roots (Table 1). Kₘ and Vₘₐₓ values were determined for a total protein extract from a single CaMV-mnanH plant as 0.21 mM MUN and 476 nmol MU/min/mg protein respectively. The in-vitro half-life of mNAN was determined in CaMV-mnanH extracts and was found to be at least 1 month for extracts stored at 4° C., −20° C. and −70° C. and approximately 60 h at room temperature. A crude estimation of the in-vivo half-life of mNAN was obtained by placing an rbcS-mnanH plant in the dark (based on the assumption that this is known to eliminate transcription from the rbcS promoter) and monitoring the declining levels of mNAN activity over time. The in vivo half-life, determined in this way was approximately 60 h. No endogenous background NAN activity was detected in leaf, stem or root extracts prepared from untransformed Arabidopsis, tobacco, rice, tomato or soybean plants.

TABLE 1
Activity of mNAN (nmol MU/min/mg protein) in tissue extracts.

| Gene construct | Leaf (c. 5 cm) | Root |
|---|---|---|
| CaMV-mnanH | 236 | 371 |
| rbcS-mnanH | 480 | 77 |

In situ Detection of GUS and mNAN Following PAGE

Because of the magnitude of the molecular weight difference (25 kDa) between GUS (68 kDa) and mNAN (43 kDa), both enzymes can be readily separated by non-denaturing PAGE or denaturing SDS-PAGE. Moreover, mNAN and GUS activities can be readily detected in situ in polyacrylamide gels following electrophoretic separation. Both enzyme activities were detectable following separation by SDS-PAGE (with sample denaturation at 60° C.) and subsequent renaturation (10, 1), or alternatively by non-denaturing PAGE, by incubating the polyacrylamide gel in GEB buffer containing both MUG and MUN (FIG. 5). Both activities were also detected in polyacrylamide gels using the histochemical substrates X-GlucM and X-NeuAc (data not shown).

Histochemical Detection of mNAN and GUS Activity in Tobacco

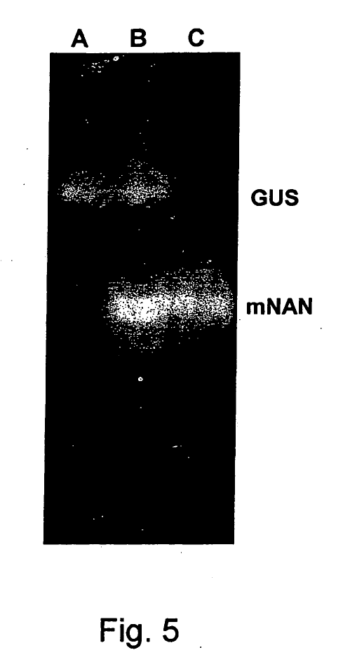

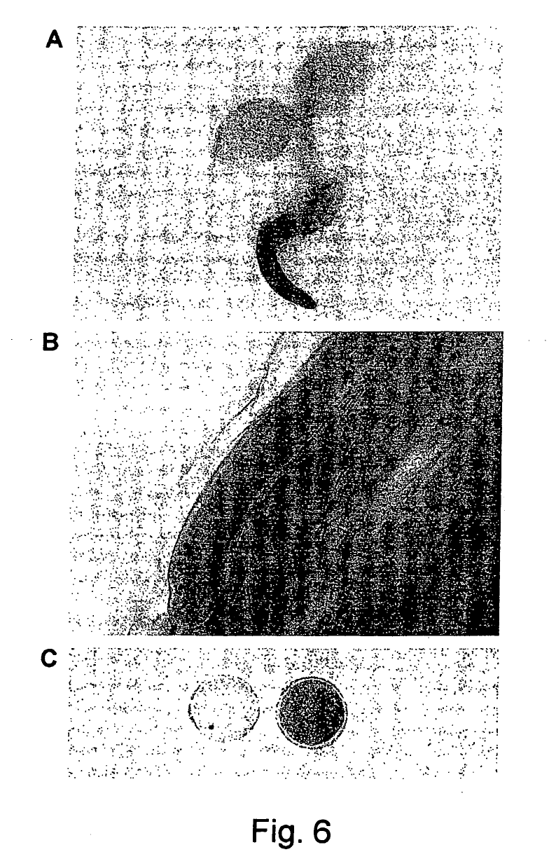

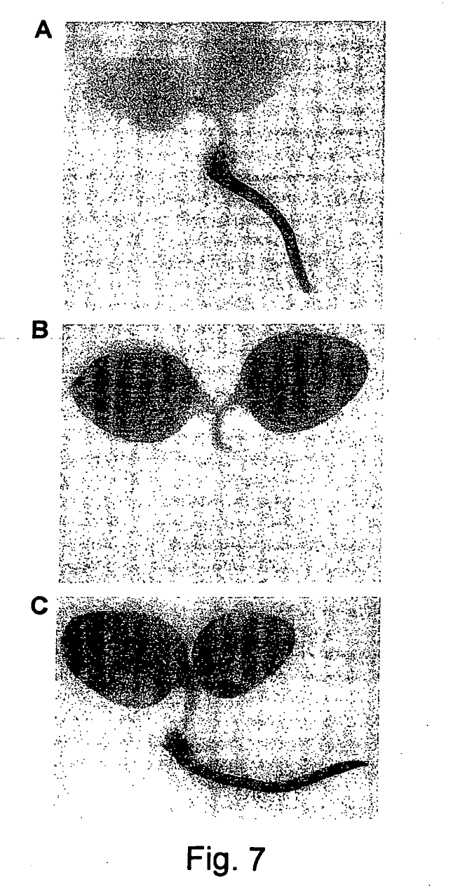

Tobacco plants expressing mnanH from the CaMV and rbcs promoters were grown to seed. mNAN activity was visualized in whole seedlings and in various tissues of these transgenic plants using the histochenical substrate X-NeuNAc (FIG. 6). Seeds produced by self-fertilization of individual CaMV-mnanH and rbcS-mnanH transgenic lines were germinated and mNAN and GUS activity determined in several seedlings of each line as shown in Table 2 below. GUS activity was determined in seedlings harbouring the GUS gene driven by one of two different promoters: CAB (17), or Bmy (18) (Table 2). In order to generate mNAN×GUS progeny (i.e. progeny containing both reporter gene activities), parental plants were chosen which expressed similar levels of mNAN or GUS enzymatic activity. These were grown to maturity and cross-pollinated. Seeds from these crosses were germinated on 1% agar plates and whole seedlings were histochemically stained for both mNAN and GUS activity (FIG. 7). Localised expression of NAN and GUS was detected as blue and magenta staining, respectively, while co-expression resulted in a deep purple colour.

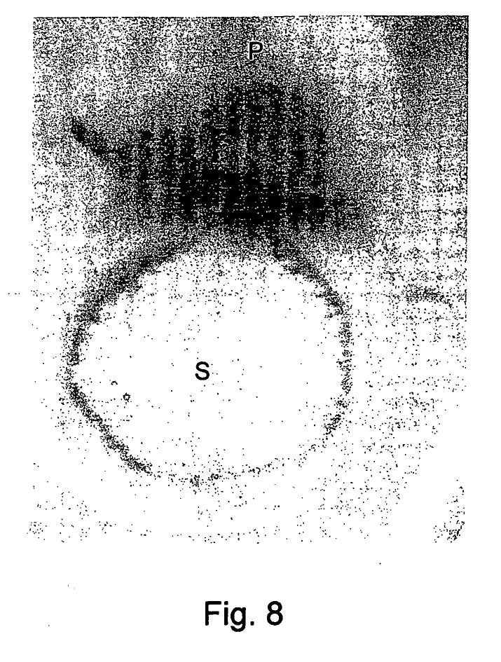

Both mNAN and GUS activities were also simultaneously localized in tissue sections from mNAN×GUS plants grown to maturity (FIG. 8).

TABLE 2

| Gene construct | Average activity |
|---|---|
| rbcS-mnanH | mNAN activity: 368 +/− 163 |
| CaMV-mnanH | mNAN activity: 200 +/− 87 |
| Bmy-GUS | GUS activity: 21 +/− 12 |
| CAB-GUS | GUS activity: 140 +/− 82 |

Simultaneous Quantitation of mNAN and GUS Activities in Cell Free Extracts

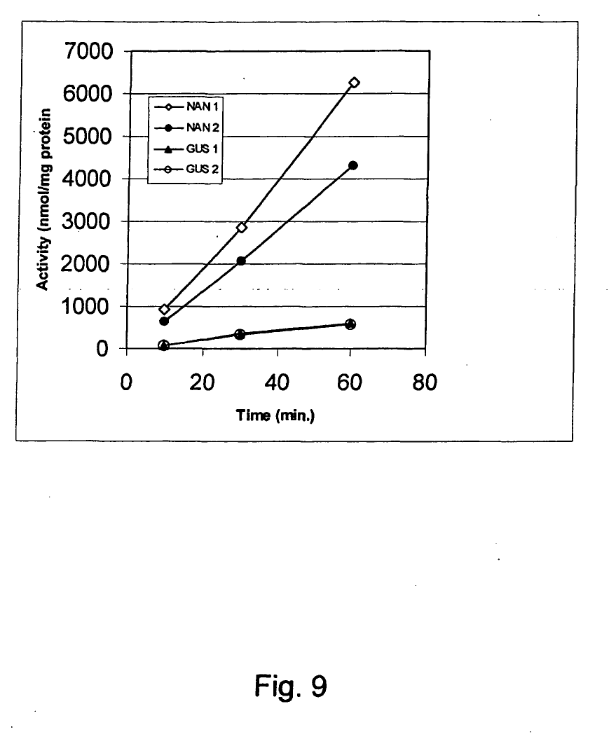

Since both mNAN and GUS can be assayed in the same buffer (GEB), it is convenient to prepare plant extracts in GEB and assay their activities in parallel using MUN and MUG. The possibility of detecting both enzyme activities in the same reaction was also investigated using resorufin β-D-glucuronide (ReG, Sigma) as a GUS substrate and MUG for detection of mNAN. Since enzymatic cleavage of both substrates releases a different fluorogenic product, it should in theory be possible to quantify the two activities in the same sample. The Kₘ for GUS activity using ReG as substrate was determined to be 0.15 mM and this concentration was used in subsequent reactions. Extracts containing both activities were assayed for mNAN and GUS independently and also simultaneously and the results compared (FIG. 9). GUS activities were equivalent whether measured alone or simultaneously with mNAN in the same sample (i.e. both reactions assayed in the same tube), but mNAN activity was apparently 30% lower when measured simultaneously with GUS as compared to alone. This discrepancy turned out to be due to absorption of light emitted by methylumbelliferone (MU) by the uncleaved ReG substrate (i.e. ReG-mediated quenching of MU fluoresence), and might be resolved by the use of a correction factor. An alternative strategy for simultaneous detection of mNAN and GUS in the same sample might be to use substrates whose light absorption and emission characteristics (and those of their cleavage products) do not overlap.

mNAN Tolerance of N- and C-terminal Fusions

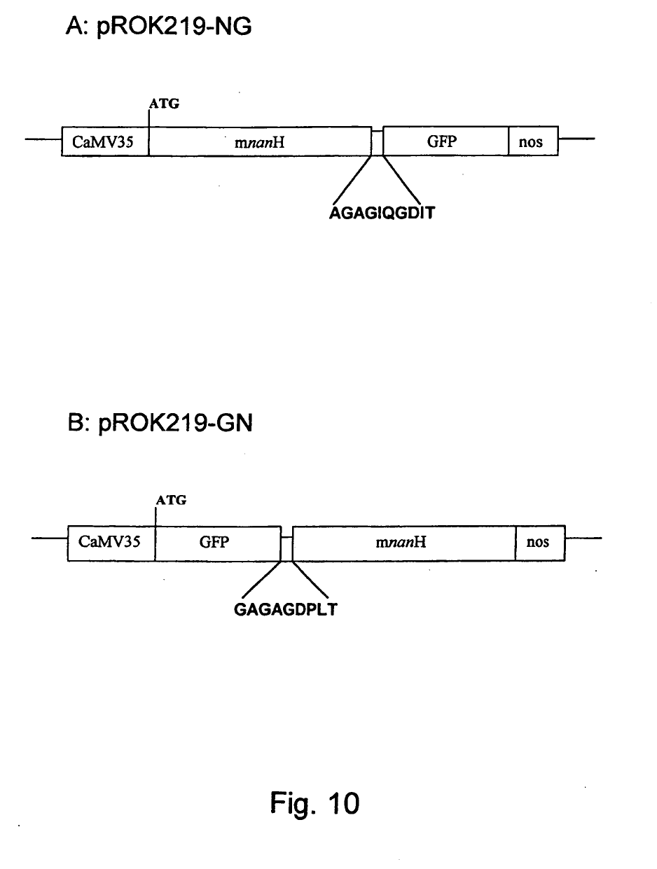

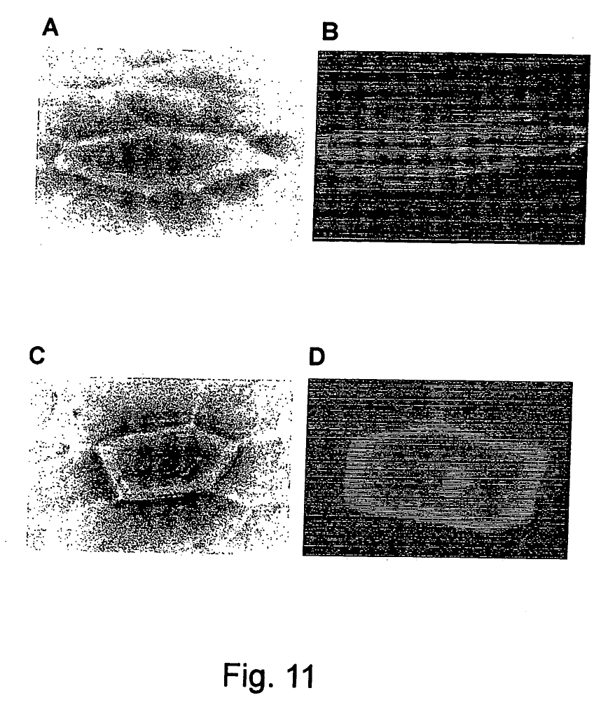

In order to investigate the ability of mNAN to function as part of a chimeric fusion protein, translational gene fusions were constructed between mnanH and the gene encoding jellyfish green fluorescent protein (GFP) (19). Two gene fusions encoding GFP-mNAN and mNAN-GFP under control of the CaMV 35S promoter in pRok219 were made (FIG. 10). Transient expression in onion epidermal cells resulted in both green fluorescence (due to GFP) and mNAN activity in targeted cells (FIG. 11).

The invention is not limited to the embodiments hereinbefore described which may be varied in detail.

1. Use of a gene which codes for a sialidase activity as a reporter gene.

2. Use of a gene as claimed in claim 1 as a reporter gene in eukaryotic organisms or cells.

3. Use of a gene as claimed in claim 1 as a reporter gene in an organelle or cell or organism having a prokaryotic-like transcriptional and translational system.

4. Use of a gene as claimed in claim 1 as a reporter gene in plants.

5. Use of a gene as claimed in claim 1 as an organelle-specific reporter gene.

6. Use of a gene as claimed in claim 1 as a reporter gene in transiently transformed prokaryotic or eukaryotic cells.

7. Use of a gene as claimed in claim 1 as a reporter gene in stably transformed prokaryotic or eukaryotic cells.

8. Use as claimed in claim 1 wherein the nucleotide sequence of the natural gene is modified.

9. Use as claimed in claim 1 wherein the gene comprises SEQ ID NO. 1.

10. Use as claimed in claim 1 wherein the gene is a modified nanH.

11. Use as claimed in claim 1 wherein the gene is isolated from a prokaryotic or eukaryotic organism.

12. Use as claimed in claim 1 wherein the gene is isolated from a bacterium.

13. Use as claimed in claim 1 wherein the gene is isolated from Clostridium perfringens.

14. Use of a gene as claimed in claim 1 as a reporter gene in combination with another reporter gene.

15. Use as claimed in claim 14 wherein the other reporter gene is the uidA gene of Escherichia coli which encodes the enzyme β-glucuronidase (GUS).

16. A gene having SEQ ID NO. 1.

17. A modified gene encoding a protein having sialidase enzymatic activity.

18. A modified gene as claimed in claim 16 whose nucleotide sequence encodes a prokaryotic or eukaryotic sialidase enzymatic activity.

19. A modified gene as claimed in claim 16 whose nucleotide sequence encodes a bacterial sialidase enzymatic activity.

20. A modified gene as claimed in claim 16 whose nucleotide sequence encodes a Clostridium perfringens sialidase activity.

21. A modified gene having SEQ ID No.1 the nucleotide sequence of which is based on the nanH gene from Clostridium perfringens which codes for a sialidase enzymatic activity.

22. A sialidase enzymatic activity encoded by a gene as claimed in claim 16.

23. Use of a gene as claimed in any of claims 16 or a derivative thereof in the production of sialidase.

24. Use of a gene as claimed in claim 16 or a derivative thereof in the production of anti-sialidase antibodies.

25. Use of a gene as claimed in claim 16 or a derivative thereof in the production of a vaccine.

26. Use of a gene as claimed in claim 16 or a derivative thereof to investigate the biological consequences of sialoglycoconjugate cleavage in animals.

27. Use of a gene as claimed in claim 16 or a derivative thereof or the encoded protein as a therapeutic agent in animals including man.

28. Use of a gene as claimed in claim 16 or a derivative thereof in the production of transgenic animals or animal cell lines.

29. Use of a gene as defined in claim 16 for quantifying or detecting sialidase (NAN) activity in cell-free extracts or in whole organisms, organs, tissues or cells or in histological sections thereof or in vivo in whole cells using fluorogenic, chromogenic or calorimetric substrates.

30. Use of a gene as defined in claim 16 for simultaneously quantifying or detecting NAN and GUS activity in cell-free extracts or in whole organisms, organs, tissues or cells or in histological sections thereof or in vivo in whole cells using fluorogenic, chromogenic, calorimetric or histochemical substrates.


SEQUENCE LISTING (1 sequence)

SEQ ID NO: 1 - DNA, 1188 bp, Artificial sequence
  Synthesized
tcgactctagaggatccgttaacaatgtgtaacaagaacaacaccttcgagaagaacctcgacatctcacacaagcctgaaccacttatcctctttaacaaggataacaacatctggaattctaagtacttcaggattcctaacatccagttgcttaatgacggtacaatccttaccttctctgacatcaggtacaacgggcccgatgaccacgcttacattgatatcgcttctgctagatctactgaattcggtaagacctggtcttacaacatcgctatgaagaacaacaggatcgactcaacctactcacgtgtgatggattctaccactgtgatcactaacaccggccggatcattcttatcgctggatcttggaacactaacggaaactgggctatgaccacttctaccagaaggtctgattggtctgtgcagatgatctactctgatgacaacggacttacttggtctaacaagatcgatctcactaaggactcttcaaaagtgaagaaccagccttctaacacaattggatggctcggaggtgttggatctggaatcgttatggacgatggaaccatcgttatgcctgctcagatctccttaagagaaaacaacgagaacaactactattcactcatcatatattccaaggataacggcgagacttggactatggggaacaaggtgccgaattccaatacgtccgagaatatggtcattgaactcgacggagcattgatcatgtctactaggtacgattactcaggctacagagcggcatacataagtcatgacctcgggacaacttgggaaatctacgagccacttaatggcaagattctcacaggcaaaggttccggatgtcaaggatcattcatcaaagccacaacgagtaatggacatcgtattggactcattagtgcacctaagaacacaaaaggtgagtacattagagataatatcgccgtctacatgatcgattttgacgatctgagcaaaggtgttcaagagatctgcattccatatccagaggatggtaacaagctcggtggaggatactcctgtctgagttttaagaacaaccacttgggtattgtttacgaagctaatggtaatatcgagtatcaggacttgacaccatactatagtcttattaacaagcagtgagagctcggtacctcg